(19) United States
(12) Patent Application Publication
(10) Pub. No.: US 2026/0076662 A1
(43) Pub. Date: Mar. 19, 2026

(54) SPINAL RETRACTOR SYSTEM

(71) Applicant: GENESYS SPINE, Austin, TX (US)

(72) Inventors: Landon GILKEY, Farmington, NM (US); Josh KAUFMANN, Austin, TX (US)

(21) Appl. No.: 19/125,074

(22) PCT Filed: Nov. 10, 2023
(86) PCT No.: PCT/US2023/079302

§ 371 Date: Apr. 28, 2025

Related U.S. Application Data
(60) Provisional application No. 63/424,156, filed on Nov. 10, 2022.
(60) Provisional application No. 63/542,396, filed on Oct. 4, 2023.

(51) Int. Cl. A61B 17/02; A61B 17/00; A61B 17/70

(52) U.S. Cl. CPC A61B 17/025; A61B 17/0206; A61B 17/7077; A61B 2017/00367; A61B 2017/00477; A61B 2017/00862; A61B 2017/0256

(57) ABSTRACT

An embodiment of a retractor system includes a retractor blade with actuating members at the blade tip, which allow the blade to fixedly couple to and decouple from the shank of a pedicle screw with the press of a button. The actuating members each have an arced slot that acts like a cam when the button is depressed. The button-based actuation is based, at least in part, on the shape of the arcs relative to the pivot point. When the blade is in the relaxed state with the button not depressed the slope of the arc aligns with the pivot point. This prevents the actuating members from being opened from forces that may be generated during ordinary screw manipulation. When the blade is in the actuated state with the button depressed the slope of the arc is offset from the pivot point. This allows the blades to move as needed.

### CROSS-REFERENCE TO RELATED APPLICATIONS

This application claims priority to U.S. Provisional Patent Application No. 63/424,156 filed on Nov. 10, 2022 and entitled “Spinal Retractor System”, the content of which is hereby incorporated by reference. This application further claims priority to U.S. Provisional Patent Application No. 63/542,396 filed on Oct. 4, 2023 and entitled “Spinal Retractor System”, the content of which is hereby incorporated by reference.

### BACKGROUND

In varying forms, retractors are used to hold an incision open and hold back tissues or other objects to maintain a clear surgical field, or reach other structures.

### BRIEF DESCRIPTION OF THE DRAWINGS

Features and advantages of embodiments of the present invention will become apparent from the appended claims, the following detailed description of one or more example embodiments, and the corresponding figures. Where considered appropriate, reference labels have been repeated among the figures to indicate corresponding or analogous elements.

FIG. 1 includes a perspective view of a retractor system in an embodiment.

FIGS. 2 and 3 include side views of a retractor blade or arm in an embodiment.

FIG. 4 includes a perspective view of the embodiment of FIGS. 2-3.

FIGS. 5-6 include side views of a portion of the embodiment of FIGS. 2-3.

FIGS. 7A and 7B include side views of a retractor blade or arm in an embodiment. FIG. 7C includes a perspective view of the embodiment of FIGS. 7A and 7B. FIGS. 7D-7E include side views of a portion of the embodiment of FIGS. 7A-7B.

FIGS. 8A and 8B include side views of a retractor blade or arm in an embodiment.

FIGS. 9A and 9B include side views of a retractor blade or arm in an embodiment. FIG. 9C includes a perspective view of the embodiment of FIGS. 9A and 9B.

FIGS. 10A-10B address forces managed via cam mechanisms of embodiments.

FIGS. 11A-11B and 12A-12B include side views of a portion of a retractor arm in an embodiment.

FIG. 13 includes a top view of a retractor arm assembly in an embodiment.

FIG. 14 includes a cross-sectional top view of retractor arm portions in an embodiment.

### DETAILED DESCRIPTION

Reference will now be made to the drawings wherein like structures may be provided with like suffix reference designations. In order to show the structures of various embodiments more clearly, the drawings included herein are diagrammatic representations of structures. Thus, the actual appearance of the fabricated structures, for example in a photo, may appear different while still incorporating the claimed structures of the illustrated embodiments (e.g., walls may not be exactly orthogonal to one another in actual fabricated devices). Moreover, the drawings may only show the structures useful to understand the illustrated embodiments. Additional structures known in the art may not have been included to maintain the clarity of the drawings. For example, not every layer of a device is necessarily shown. “An embodiment”, “various embodiments” and the like indicate embodiment(s) so described may include particular features, structures, or characteristics, but not every embodiment necessarily includes the particular features, structures, or characteristics. Some embodiments may have some, all, or none of the features described for other embodiments. “First”, “second”, “third” and the like describe a common object and indicate different instances of like objects are being referred to. Such adjectives do not imply objects so described must be in a given sequence, either temporally, spatially, in ranking, or in any other manner. “Connected” may indicate elements are in direct physical or electrical contact with each other and “coupled” may indicate elements co-operate or interact with each other, but they may or may not be in direct physical or electrical contact.

When describing the embodiments of the present disclosure, the following terms, if present, have the following meanings, unless otherwise indicated. If not otherwise defined, terms have their customary meaning in the relevant art.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

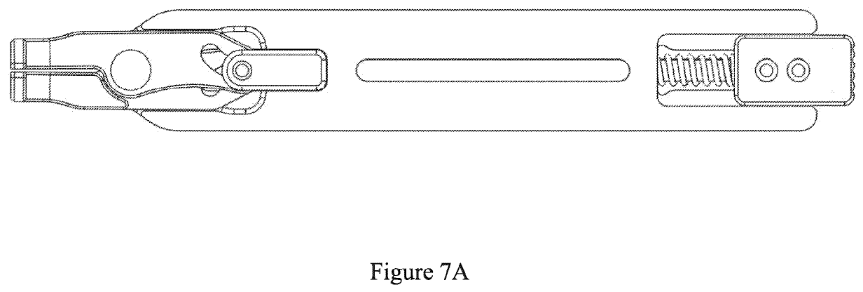

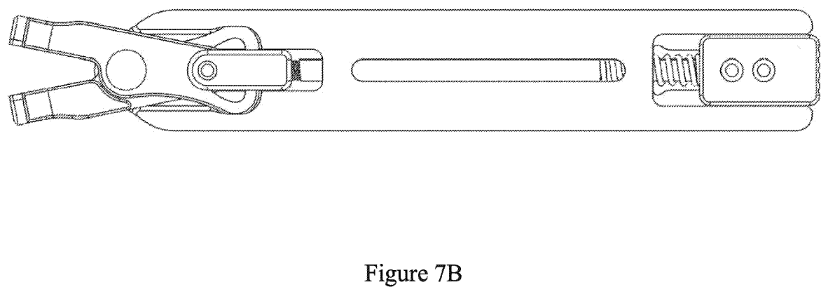

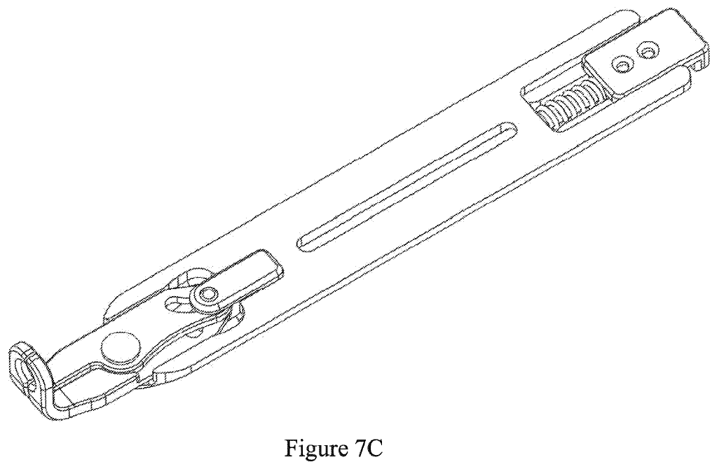

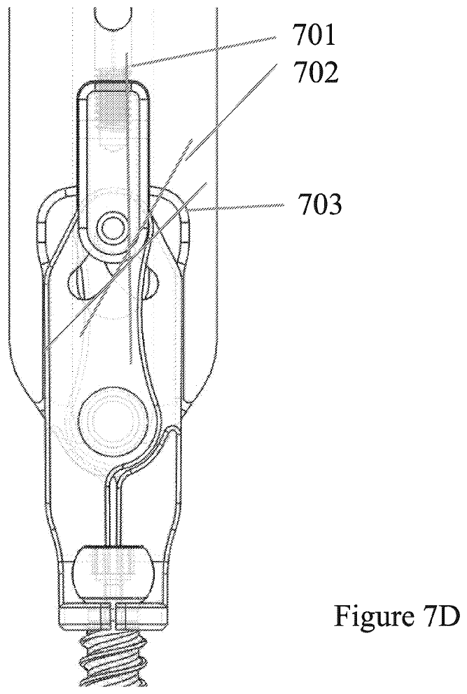

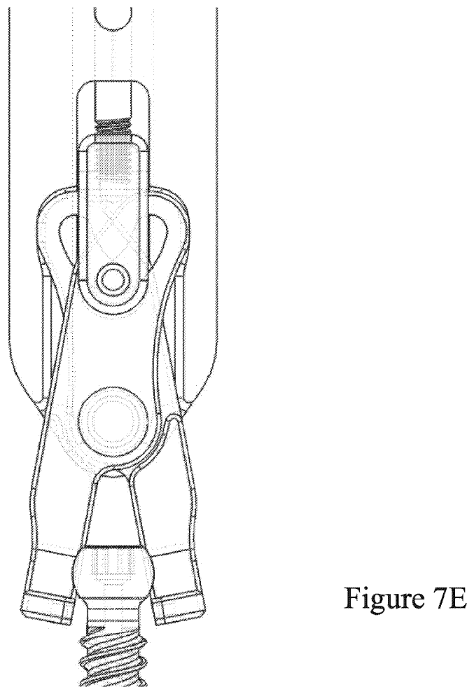

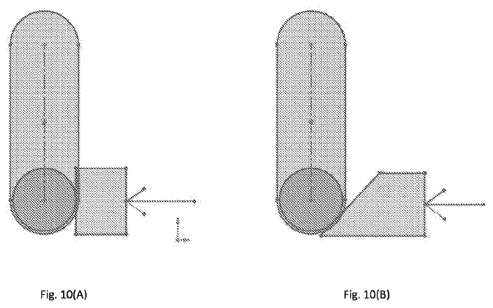

An embodiment includes a retractor system. The system includes a retractor blade with actuating members at the blade tip, which allow the blade to fixedly couple to and decouple from the shank of a pedicle screw with the press of a button. In the embodiment of FIGS. 7A-7E, the actuating members each have an arced slot that acts like a cam when the button is depressed. The button-based actuation is based, at least in part, on the shape of the arcs relative to the pivot point. When the blade is in the relaxed state with the button not depressed (the blade actuators are closed) the slope of the arc aligns with the pivot point. This prevents the actuating members from being opened from forces that may be generated during ordinary screw manipulation (e.g., distracting or compressing with the retractor). When the blade is in the actuated state with the button depressed (the blade actuators are open) the slope of the arc is offset from the pivot point. This allows the blades to move as needed. For example, line 701 is a tangent to an upper portion of an arc. Lines 702 and 703 are tangents to the arc at points further distal to the tangent point of line 701. The arcs progressively move from the more vertical orientation of line 701 to the more horizontal orientation of line 703. In doing so, the resistance decreases for opening or further spreading the actuators away from each other. As another illustration, see FIGS. 10(A) and 10(B). FIG. 10(A) is analogous to the condition associated with line 701 while FIG. 10(B) is analogous to the condition associated with line 702 or 703. Relatively vertical tangents are more resistant to actuation (i.e., vertical wedge trying to move the actuation pin) than relatively horizontal tangents (i.e., an angled wedge trying to move the actuation pin).

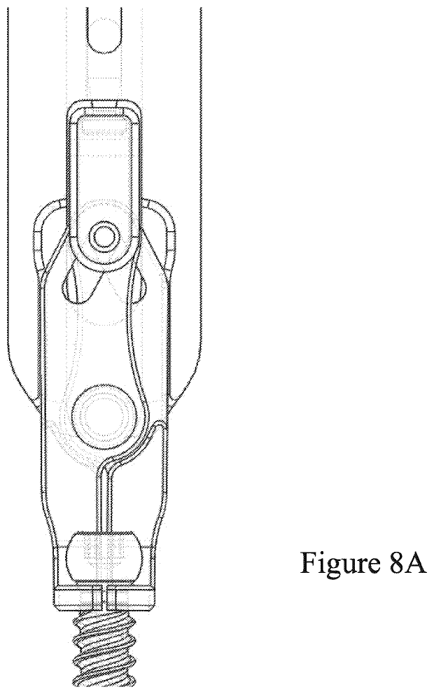

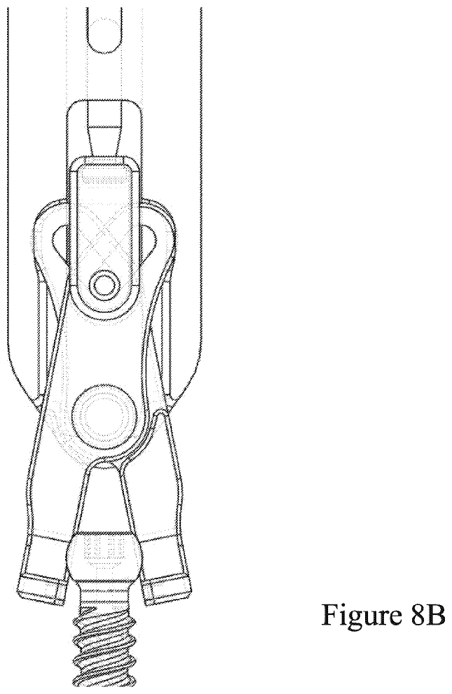

However, in other embodiments the slot may not be arced. For example, see FIGS. 8A-8B. In such a case, the actuators are still resistant to being prematurely spread apart from one another but they may be less resistant to such spreading than the embodiment of FIGS. 7A-7E.

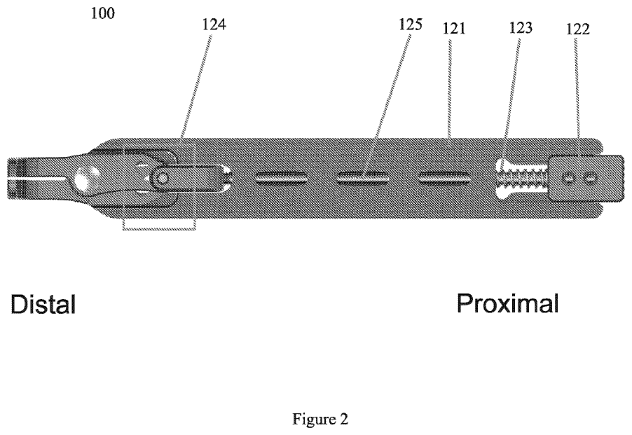

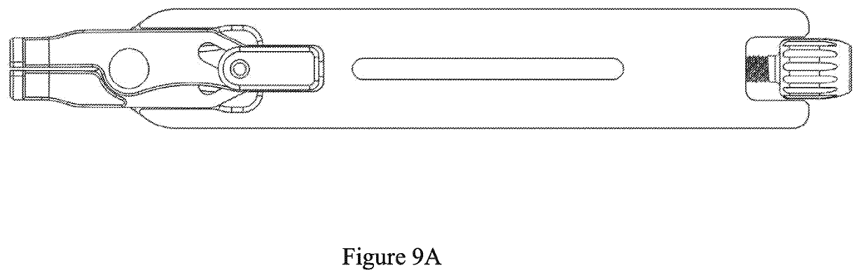

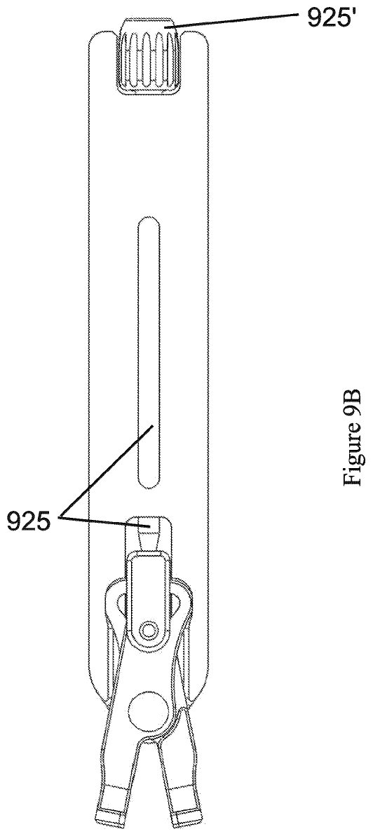

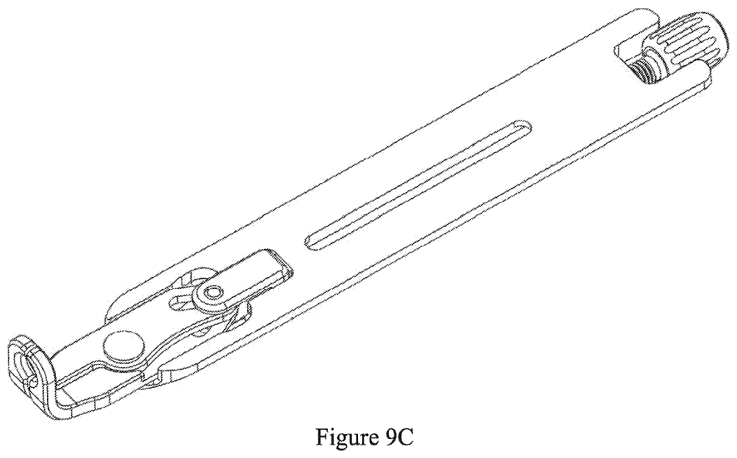

Further, some embodiments may not necessarily include resilient members and push button activation. For example, see the embodiment of FIGS. 9A-9C. In such an embodiment, loosening or tightening knob 925′ (at the proximal end of the device) to threaded rod 925 will necessarily spread the actuators away from each other or bring them closer to one another by driving rod 925 forwards (distally)/backwards (proximally). Other embodiments may reverse the relation such that tightening or loosening the knob at the proximal end of the device will necessarily spread the actuators away from each other or bring them closer to one another. In cases such as FIG. 2 or 9B, the cam is still operated in reaction to a rod (e.g., rods 125, 925) or other member being driven distally/proximally via pushing the push button, tightening or loosening a member threadingly coupled to the rod, and the like.

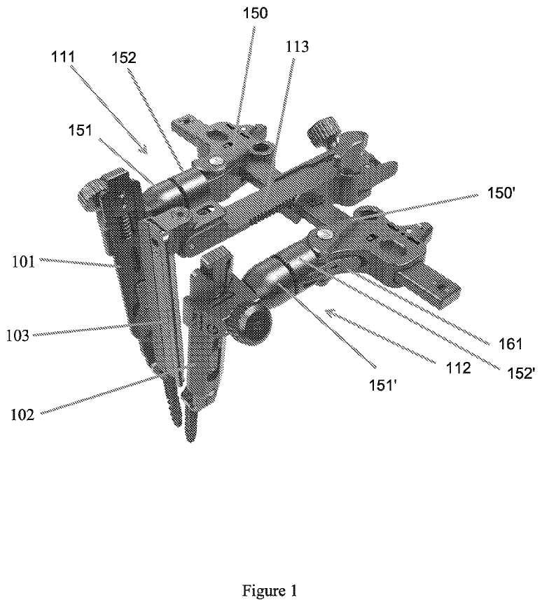

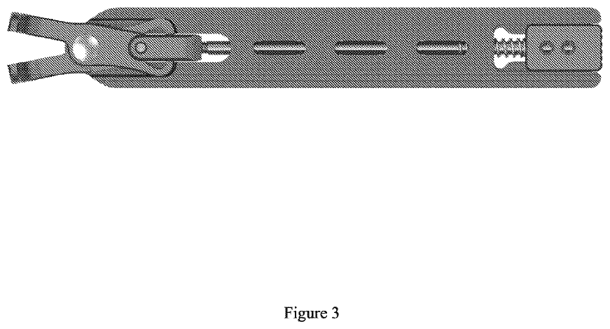

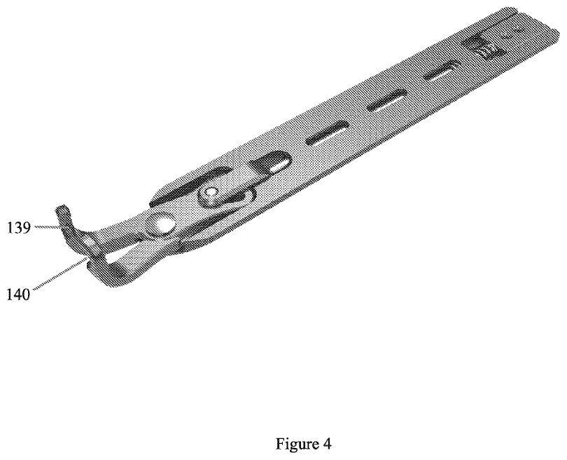

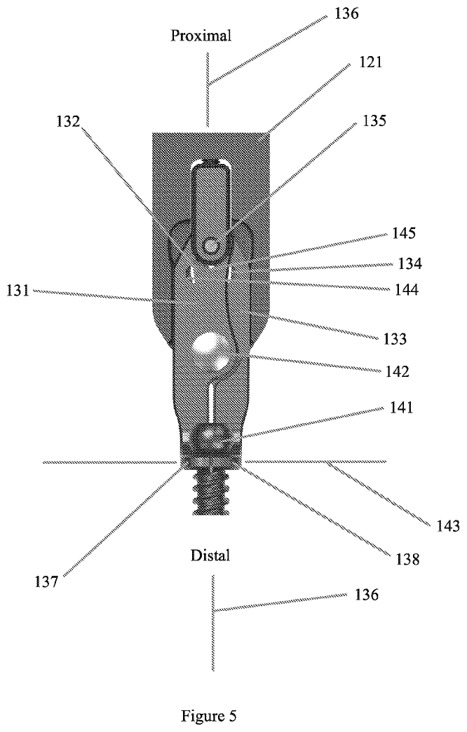

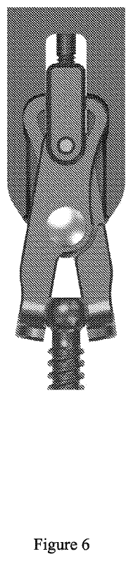

As shown in FIGS. 1-6, an embodiment includes a surgical retractor system (100). The system comprises a retractor body coupled to first, second, and third retractor arms. A first retractor blade (101) is coupled to the first retractor arm (111). A second retractor blade (102) is coupled to the second retractor arm (112). A third retractor blade (103) is coupled to the third retractor arm (113). The first retractor blade includes a blade body (121), a first button (122), a resilient member (123) coupling the first button to the blade body. The first retractor blade further includes a cam (124). The cam includes a first cam plate (131) including a first cam slot (132). The cam also includes a second cam plate (133) including a second cam slot (134). The cam further includes a cam pin (135) included within the first and second cam slots. A rod (125) couples the first button to the cam. As shown in FIG. 3, the first blade is configured so depressing the first button drives the cam pin distally along a long axis (136) of the first retractor blade. In response to the cam pin being driven distally the cam pin travels simultaneously along the first and second cam slots. In response to the cam pin traveling simultaneously along the first and second cam slots, distal portions of the first and second cam plates move away from one another.

In an embodiment shown in FIG. 2, the first blade is configured so releasing the first button drives the cam pin proximally along the long axis of the first retractor blade. In response to the cam pin being driven proximally the cam pin travels simultaneously along the first and second cam slots. In response to the cam pin traveling simultaneously along the first and second cam slots, distal portions of the first and second cam plates move towards one another. In the embodiment of FIG. 5, the first cam plate includes a first foot portion (137) and the second cam plate includes a second foot portion (138). The first foot portion includes a first void (139) and the second foot portion includes a second void (140). The first and second voids are configured to collectively surround a portion of a pedicle screw (141) when the first button is released and the distal portions of the first and second cam plates move towards one another.

The embodiment of FIG. 5 includes a pivot pin (142), wherein the first and second cam plates are pivotally coupled to one another via the pivot pin. The first foot is included in a plane (143) that is orthogonal to the long axis of the first retractor blade. The first cam slot includes opposing ends (e.g., end 144) coupled to one another by sidewalls (e.g., sidewall 145) that are primarily curved.

The second cam slot includes opposing ends coupled to one another by sidewalls that are primarily curved. For example, see FIGS. 7A-7E. The curved nature of the slot sidewalls helps prevent the actuating members from being opened from forces that may be generated during ordinary screw manipulation (e.g., distracting or compressing with the retractor).

In an embodiment, the first cam slot includes opposing ends coupled to one another by sidewalls that are primarily linear. The second cam slot includes opposing ends coupled to one another by sidewalls that are primarily linear. For example, see FIGS. 8A-8B.

Various locking mechanisms may be employed to help lock the cam plates in place and help prevent the actuating members from being opened from forces that may be generated during ordinary screw manipulation (e.g., distracting or compressing with the retractor). For example, a sleeve may be lowered over the plates to limit their lateral movement or the rod that couples to the cam pin may be locked in place. For example, a proximal end of the rod may include a void through which a dowel may be inserted or the proximal end of the rod may be threaded and locked in place with a nut or bolt.

In an embodiment, the first cam slot extends laterally as the first cam slot extends distally and the second cam slot extends laterally as the second cam slot extends distally. The first and second cam slots extend in opposing directions as the first and second cam slots extend distally and laterally. See, e.g., FIGS. 7A and 8A.

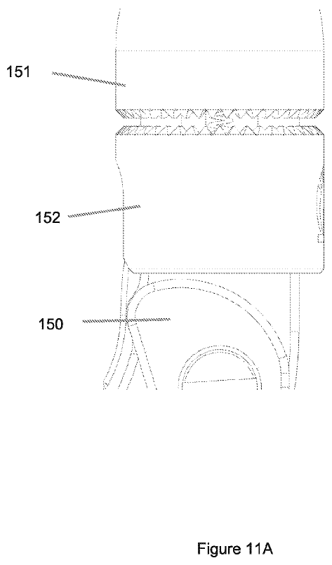

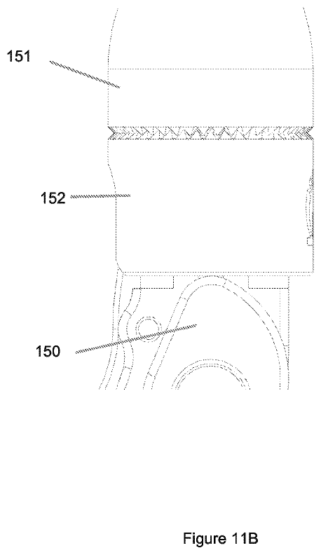

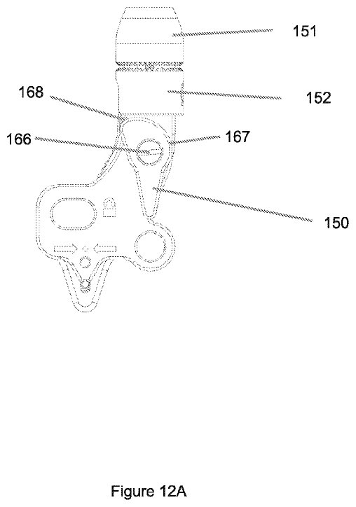

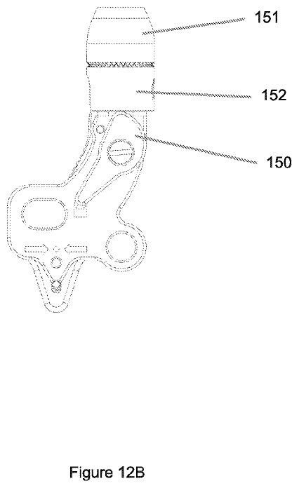

In the embodiment of FIGS. 11A and 12A, the first retractor arm includes a lever (150) and first and second portions (151, 152). The first and second portions are rotationally coupled to each other. The first portion includes an arcuate row of first teeth and the second portion includes an arcuate row of second teeth keyed to securely couple with the first teeth. In a first retractor arm orientation: (a) the lever does not directly contact the first portion, and (b) the first teeth do not directly contact the second teeth. In a second retractor arm orientation: (a) the lever directly contacts the first portion, and (b) the first teeth directly contact the second teeth to prevent rotation of the first and second portions with regard to each other. For example, FIGS. 11A, 12A illustrate the first retractor arm orientation and FIGS. 11B, 12B illustrate the second retractor arm orientation.

In an embodiment, the first retractor arm includes first and second portions (151, 152). The first and second portions are rotationally coupled to each other. The first portion includes an arcuate row of first teeth and the second portion includes an arcuate row of second teeth keyed to securely couple with the first teeth. In a first retractor arm orientation the first teeth do not directly contact the second teeth and in a second retractor arm orientation the first teeth directly contact the second teeth to prevent rotation of the first and second portions with regard to each other. Thus, in an embodiment lever 150 is not necessarily required. For example, element 152 may be threadingly coupled to arm 111. Element 152 may be then be advanced toward element 151 (by screw/unscrewing via the threaded coupling) to lock element 151 in place or advanced away from element 151 to unlock element 151. For example, element 151 may be threadingly coupled to post 162 or some other portion of the system. Element 151 may be then be advanced toward element 152 to lock element 151 in place or advanced away from element 152 to unlock element 151.

In an embodiment the first retractor arm includes a first actuator (150) and first and second portions (151, 152). The first and second portions are rotationally coupled to each other. The first portion includes an arcuate row of first teeth and the second portion includes an arcuate row of second teeth keyed to securely couple with the first teeth. In a first retractor arm orientation: (a) the first actuator does not directly contact the first portion, and (b) the first teeth do not directly contact the second teeth. In a second retractor arm orientation: (a) the first actuator directly contacts the first portion, and (b) the first teeth directly contact the second teeth to prevent rotation of the first and second portions with regard to each other. Thus, an actuator including one or more parts may be used instead of the above-mentioned lever. As used herein, An actuator is a component of a machine that is responsible for moving and controlling a mechanism or system by, for example, controlling the ability of elements 151, 152 to rotate with respect to each other. An actuator may include, for example, a sliding bar, linkage, nut, fastener, and the like that can be manipulated to control the interaction between elements 150, 151.

In an embodiment the first retractor arm includes a first actuator (150) and first and second portions (151, 152). The first and second portions are rotationally coupled to each other. The first portion includes a first interface that is keyed to a second interface of the second portion. In a first retractor arm orientation: (a) the first actuator does not directly contact the first portion, and (b) the first interface does not directly contact the second interface. In a second retractor arm orientation: (a) the first actuator directly contacts the first portion, and (b) the first interface directly contacts the second interface to prevent rotation of the first and second portions with regard to each other. Thus, toothed interfaces are not mandatory in all embodiments.

In an embodiment, the first portion is configured to rotate at least 90 degrees with respect to the second portion.

In the embodiment of FIG. 1, the second retractor arm includes an additional lever (150′) and additional first and second portions (151′, 152′). The additional first and second portions are rotationally coupled to each other. The additional first portion includes an additional arcuate row of additional first teeth and the additional second portion includes an additional arcuate row of additional second teeth keyed to securely couple with the additional first teeth. In an additional first retractor arm orientation: (a) the additional lever does not directly contact the additional first portion, and (b) the additional first teeth do not directly contact the additional second teeth. In an additional second retractor arm orientation: (a) the additional lever directly contacts the additional first portion, and (b) the additional first teeth directly contact the additional second teeth to prevent rotation of the additional first and second portions with regard to each other. In other words, the first and second arms 111, 112 have symmetrical functionality. While this illustration uses “lever” the symmetry is equally applicable to versions of example 9 including an actuator and the like.

In the embodiment of FIG. 12A, the first actuator includes a pivot point (166) about which the first actuator pivots. The first actuator has a perimeter and a first point (167) on the perimeter is closer to the pivot point than a second point (168) on the perimeter.

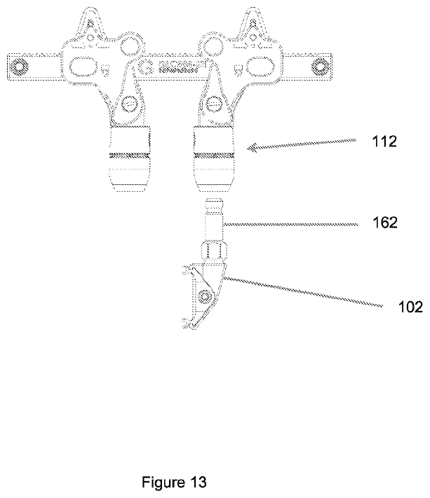

In an embodiment, the second retractor arm is releasably coupled to the second retractor blade. For example, FIG. 13 illustrates the second retractor arm released or separated from the second retractor blade whereas FIG. 1 illustrates the second retractor arm coupled to the second retractor blade.

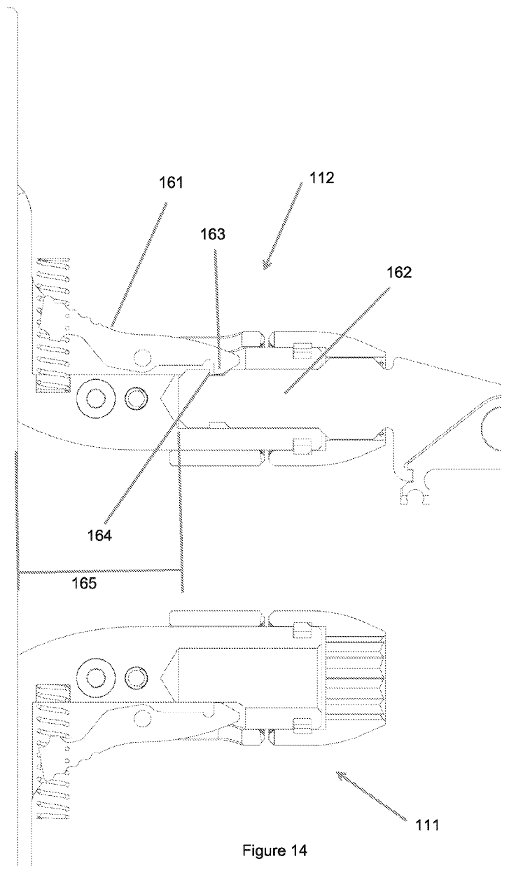

An embodiment includes a second button (161). In a first blade orientation: (a) the second button is not depressed, and (b) the second retractor arm is locked to the second retractor blade. In a second blade orientation the second retractor arm is not locked to the second retractor blade. For example, FIG. 14 illustrates the first blade orientation with the second button not depressed, and the second retractor arm is locked to the second retractor blade. FIG. 13 illustrates the second blade orientation with the second retractor arm not locked to the second retractor blade.

In an embodiment in a first blade orientation: (a) the second actuator is unactuated, and (b) the second retractor arm is locked to the second retractor blade. In a second blade orientation the second retractor arm is not locked to the second retractor blade. In the second blade orientation: (a) the second button is depressed, and (b) the second retractor arm is not locked to the second retractor blade. For example, this may occur while sliding the second retractor blade post 162 away from second retractor arm 112.

In an embodiment in the second blade orientation: (a) the second actuator is actuated, and (b) the second retractor arm is not locked to the second retractor blade.

In an embodiment in the second blade orientation the second retractor arm is slidingly engaged with the second retractor blade. For example, this may occur while sliding the second retractor blade post 162 away from second retractor arm 112. However, FIG. 13 also illustrates the second blade orientation whereby the second retractor blade is fully separated from the second retractor arm. FIG. 14 shows the first retractor arm.

An embodiment comprises a resilient member coupled to the second button. The resilient member biases the second button into the first blade orientation.

An embodiment includes an additional second button, wherein: in an additional first blade orientation: (a) the additional second button is not depressed, and (b) the first retractor arm is locked to the first retractor blade. In an additional second blade orientation the first retractor arm is not locked to the first retractor blade. In other words, the first and second arms/blade systems have symmetrical functionality whereby buttons or actuators may be used to detach blades from their respective arms. While this illustration uses “button” the symmetry is equally applicable to embodiments described as including an actuator and the like.

An embodiment includes a second actuator and a protuberance (163) keyed to a void (164). The second actuator includes one of the protuberance or the void and the second retractor blade includes another of the protuberance or the void. In the first blade orientation the protuberance is located in the void.

An embodiment includes a second actuator and a protuberance and a plurality of voids. The second actuator includes one of the protuberance or the plurality of voids and the second retractor blade includes another of the protuberance or the plurality of voids. In the first blade orientation the protuberance is located in one of the plurality of voids. For example, portion 162 may include the plurality of voids and distance 165 may vary depending on which of the voids the protuberance is lodged. This may give the operator greater flexibility in positioning parts of the system with respect to the patient.

An embodiment includes a second actuator and a plurality of protuberances and a void. The second actuator includes one of the plurality of protuberances or the void and the second retractor blade includes another of the plurality of protuberances or the void. In the first blade orientation one of the plurality of protuberances is located in the void.

### EXAMPLES

### Example 1a

A surgical retractor system comprising: a retractor body coupled to first and second retractor arms; a first retractor member coupled to the first retractor arm and a second retractor member coupled to the second retractor arm; wherein the first retractor member includes: a member body; a resilient member coupled to the member body; a first plate including a first slot, a second plate including a second slot, and a pin included within the first and second slots; a rod coupling the resilient member to the pin; wherein first retractor member is configured so: (a) depressing the resilient member drives the pin distally, (b) in response to the pin being driven distally the pin travels simultaneously along the first and second slots; and (c) in response to the pin traveling simultaneously along the first and second slots, distal portions of the first and second plates move away from one another.

### Example 2a

The surgical retractor system of example 1a, wherein first retractor member is configured so: (a) releasing the resilient member drives the pin proximally, (b) in response to the pin being driven proximally the pin travels simultaneously along the first and second slots; and (c) in response to the pin traveling simultaneously along the first and second cam slots, distal portions of the first and second plates move towards one another.

### Example 3a

The surgical retractor system according to any of examples 1a-2a, wherein: the first plate includes a first portion and the second cam plate includes a second portion; the first portion includes a first void and the second portion includes a second void; the first and second voids are configured to collectively surround a portion of a pedicle screw when the resilient member is released and the distal portions of the first and second plates move towards one another.

### Example 4a

The surgical retractor system according to any of examples 1a-3a comprising a pivot pin, wherein the first and second plates are pivotally coupled to one another via the pivot pin.

### Example 5a

The surgical retractor system according to any of examples 3a-4a, wherein the first portion is included in a plane that is orthogonal to a long axis of the first retractor member.

### Example 6a

The surgical retractor system according to any of examples 1a-5a, wherein: the first slot includes opposing ends coupled to one another by sidewalls that are primarily curved; the second slot includes opposing ends coupled to one another by sidewalls that are primarily curved.

### Example 7a

The surgical retractor system according to any of examples 1a-5a, wherein: the first slot includes opposing ends coupled to one another by sidewalls that are primarily linear; the second slot includes opposing ends coupled to one another by sidewalls that are primarily linear.

### Example 8a

The surgical retractor system according to any of examples 1a-7a, wherein: the first slot extends laterally as the first slot extends distally; the second slot extends laterally as the second slot extends distally; the first and second slots extend in opposing directions as the first and second slots extend distally and laterally.

### Example 1b

A surgical retractor system comprising: a retractor body coupled to first and second retractor arms; a first retractor member coupled to the first retractor arm and a second retractor member coupled to the second retractor arm; wherein the first retractor member includes: a member body; a first plate including a first slot, a second plate including a second slot, and a pin included within the first and second slots; a rod coupling the member body to the pin; wherein first retractor member is configured so: (a) driving the rod distally drives the pin distally, (b) in response to the pin being driven distally the pin travels simultaneously along the first and second slots; and (c) in response to the pin traveling simultaneously along the first and second slots, distal portions of the first and second plates move away from one another.

Thus, not all embodiments necessarily require a resilient member. For example, see FIGS. 9A-9C.

### Example 2b

The surgical retractor system of example 1b, wherein first retractor member is configured so: (a) driving the rod proximally drives the pin proximally, (b) in response to the pin being driven proximally the pin travels simultaneously along the first and second slots; and (c) in response to the pin traveling simultaneously along the first and second cam slots, distal portions of the first and second plates move towards one another.

### Example 3b

The surgical retractor system according to any of examples 1b-2b, wherein: the first plate includes a first portion and the second cam plate includes a second portion; the first portion includes a first void and the second portion includes a second void; the first and second voids are configured to collectively surround a portion of a pedicle screw when the rod is driving proximally and the distal portions of the first and second plates move towards one another.

### Example 4b

The surgical retractor system according to any of examples 1b-3b comprising a pivot pin, wherein the first and second plates are pivotally coupled to one another via the pivot pin.

### Example 5b

The surgical retractor system according to any of examples 3b-4b, wherein the first portion is included in a plane that is orthogonal to a long axis of the first retractor member.

### Example 6b

The surgical retractor system according to any of examples 1b-5b, wherein the first slot is curved and the second slot is curved.

### Example 6b

The surgical retractor system according to any of examples 1b-5b, wherein: the first slot includes opposing ends coupled to one another by sidewalls that are primarily curved; the second slot includes opposing ends coupled to one another by sidewalls that are primarily curved.

### Example 7b

The surgical retractor system according to any of examples 1b-5b, wherein: the first slot includes opposing ends coupled to one another by sidewalls that are primarily linear; the second slot includes opposing ends coupled to one another by sidewalls that are primarily linear.

### Example 8b

The surgical retractor system according to any of examples 1b-7b, wherein: the first slot extends laterally as the first slot extends distally; the second slot extends laterally as the second slot extends distally; the first and second slots extend in opposing directions as the first and second slots extend distally and laterally.

### Example 1c

A surgical retractor system comprising: a retractor body coupled to first and second retractor arms; a first retractor member coupled to the first retractor arm and a second retractor member coupled to the second retractor arm; wherein the first retractor member includes: a member body; a resilient member coupled to the member body; a first plate including a first slot and a pin included within the first slot; a rod coupling the resilient member to the pin; wherein first retractor member is configured so: (a) depressing the resilient member drives the pin distally, (b) in response to the pin being driven distally the pin travels along the first slot; and (c) in response to the pin traveling along the first slot, a distal portion of the first plate moves laterally away from a long axis of the first retractor member.

Thus, not all embodiments require two plates that move away from each other.

### Example 2c

The surgical retractor system of example 1c, wherein first retractor member is configured so: (a) releasing the resilient member drives the pin proximally, (b) in response to the pin being driven proximally the pin travels along the first slot; and (c) in response to the pin traveling along the first slot, the distal portion of the first plate moves medially towards the long axis of the first retractor member.

### Example 3c

The surgical retractor system according to any of examples 1c-2c, wherein: the first plate includes a first portion; the first portion includes a first void; the first void surrounds a portion of a pedicle screw when the resilient member is released and the distal portion of the first plate moves medially towards the long axis of the first retractor member.

### Example 4c

The surgical retractor system according to any of examples 1c-3c comprising a pivot pin, wherein the first plate is pivotally coupled to the pivot pin.

### Example 5c

The surgical retractor system according to any of examples 3c-4c, wherein the first portion is included in a plane that is orthogonal to the long axis of the first retractor member.

### Example 6c

The surgical retractor system according to any of examples 1c-5c, wherein: the first slot includes opposing ends coupled to one another by sidewalls that are primarily curved.

### Example 7c

The surgical retractor system according to any of examples 1c-5c, wherein: the first slot includes opposing ends coupled to one another by sidewalls that are primarily linear.

### Example 8c

The surgical retractor system according to any of examples 1c-7c, wherein: the first slot extends laterally as the first slot extends distally.

### Example 1d

A surgical retractor system (100) comprising: a retractor body coupled to first, second, and third retractor arms; a first retractor blade (101) coupled to the first retractor arm (111), a second retractor blade (102) coupled to the second retractor arm (112), and a third retractor blade (103) coupled to the third retractor arm (113); wherein the second retractor arm is releasably coupled to the second retractor blade.

### Example 2d

The surgical retractor system of example 1d comprising a second button (161), wherein: in a first blade orientation: (a) the second button is not depressed, and (b) the second retractor arm is locked to the second retractor blade; in a second blade orientation the second retractor arm is not locked to the second retractor blade.

As used herein for example set “d” (e.g., Example 1d, 2d, etc.), a “second button” does not necessarily mean the system includes a “first button” although in some embodiments a “first button”is included in the system.

### Example 3d

The surgical retractor system of example 2d, wherein in the second blade orientation: (a) the second button is depressed, and (b) the second retractor arm is not locked to the second retractor blade.

### Example 4d

The surgical retractor system of example 1d comprising a second actuator, wherein: in a first blade orientation: (a) the second actuator is unactuated, and (b) the second retractor arm is locked to the second retractor blade; in a second blade orientation the second retractor arm is not locked to the second retractor blade.

### Example 5d

The surgical retractor system according to example 4d, wherein in the second blade orientation: (a) the second actuator is actuated, and (b) the second retractor arm is not locked to the second retractor blade.

### Example 6d

The surgical retractor system according to any of examples 2d-5d wherein in the second blade orientation the second retractor arm is slidingly engaged with the second retractor blade.

### Example 7d

The surgical retractor system according to any of examples 2d-3d comprising a resilient member coupled to the second button, wherein the resilient member biases the second button into the first blade orientation.

### Example 8d

The surgical retractor system of example 7d comprising an additional second button, wherein: in an additional first blade orientation: (a) the additional second button is not depressed, and (b) the first retractor arm is locked to the first retractor blade; in an additional second blade orientation the first retractor arm is not locked to the first retractor blade.

### Example 9d

The surgical retractor system according to example 1d comprising: a second actuator and a protuberance (163) keyed to a void (164); the second actuator includes one of the protuberance or the void and the second retractor blade includes another of the protuberance or the void; in a first blade orientation the protuberance is located in the void.

### Example 10d

The surgical retractor system according to example 1d comprising: a second actuator and a protuberance and a plurality of voids; the second actuator includes one of the protuberance or the plurality of voids and the second retractor blade includes another of the protuberance or the plurality of voids; in a first blade orientation the protuberance is located in one of the plurality of voids.

### Example 11d

The surgical retractor system according to example 1d comprising: a second actuator and a plurality of protuberances and a void; the second actuator includes one of the plurality of protuberances or the void and the second retractor blade includes another of the plurality of protuberances or the void; in a first blade orientation one of the plurality of protuberances is located in the void.

### Example 12d

The surgical retractor system according to example 1d, wherein: the first retractor arm includes a lever (150) and first and second portions (151, 152); the first and second portions are rotationally coupled to each other; the first portion includes an arcuate row of first teeth and the second portion includes an arcuate row of second teeth keyed to securely couple with the first teeth; in a first retractor arm orientation: (a) the lever does not directly contact the first portion, and (b) the first teeth do not directly contact the second teeth; in a second retractor arm orientation: (a) the lever directly contacts the first portion, and (b) the first teeth directly contact the second teeth to prevent rotation of the first and second portions with regard to each other.

### Example 13d

The surgical retractor system according to example 1d, wherein: the first retractor arm includes first and second portions (151, 152); the first and second portions are rotationally coupled to each other; the first portion includes an arcuate row of first teeth and the second portion includes an arcuate row of second teeth keyed to securely couple with the first teeth; in a first retractor arm orientation the first teeth do not directly contact the second teeth; in a second retractor arm orientation the first teeth directly contact the second teeth to prevent rotation of the first and second portions with regard to each other.

### Example 14d

The surgical retractor system according to example 1d, wherein: the first retractor arm includes a first actuator (150) and first and second portions (151, 152); the first and second portions are rotationally coupled to each other; the first portion includes an arcuate row of first teeth and the second portion includes an arcuate row of second teeth keyed to securely couple with the first teeth; in a first retractor arm orientation: (a) the first actuator does not directly contact the first portion, and (b) the first teeth do not directly contact the second teeth; in a second retractor arm orientation: (a) the first actuator directly contacts the first portion, and (b) the first teeth directly contact the second teeth to prevent rotation of the first and second portions with regard to each other.

### Example 15d

The surgical retractor system according to example 1d, wherein: the first retractor arm includes a first actuator (150) and first and second portions (151, 152); the first and second portions are rotationally coupled to each other; the first portion includes a first interface that is keyed to a second interface of the second portion; in a first retractor arm orientation: (a) the first actuator does not directly contact the first portion, and (b) the first interface does not directly contact the second interface; in a second retractor arm orientation: (a) the first actuator directly contacts the first portion, and (b) the first interface directly contacts the second interface to prevent rotation of the first and second portions with regard to each other.

### Example 16d

The surgical retractor system according to any of examples 12d-15d, wherein the first portion is configured to rotate at least 90 degrees with respect to the second portion.

### Example 17d

The surgical retractor system according to example 15d, wherein: the second retractor arm includes an additional lever (150′) and additional first and second portions (151′, 152′); the additional first and second portions are rotationally coupled to each other; the additional first portion includes an additional arcuate row of additional first teeth and the additional second portion includes an additional arcuate row of additional second teeth keyed to securely couple with the additional first teeth; in an additional first retractor arm orientation: (a) the additional lever does not directly contact the additional first portion, and (b) the additional first teeth do not directly contact the additional second teeth; in an additional second retractor arm orientation: (a) the additional lever directly contacts the additional first portion, and (b) the additional first teeth directly contact the additional second teeth to prevent rotation of the additional first and second portions with regard to each other.

### Example 18d

The surgical retractor system according to any of examples 14d-15d, wherein the first actuator includes a pivot point (166) about which the first actuator pivots; the first actuator has a perimeter, a first point (167) on the perimeter is closer to the pivot point than a second point (168) on the perimeter.

The foregoing description of the embodiments of the invention has been presented for the purposes of illustration and description. It is not intended to be exhaustive or to limit the invention to the precise forms disclosed. This description and the claims following include terms, such as left, right, top, bottom, over, under, upper, lower, first, second, etc. that are used for descriptive purposes only and are not to be construed as limiting. For example, terms designating relative vertical position refer to a situation where a side of a substrate is the “top” surface of that substrate; the substrate may actually be in any orientation so that a “top” side of a substrate may be lower than the “bottom” side in a standard terrestrial frame of reference and still fall within the meaning of the term “top.” The term “on” as used herein (including in the claims) does not indicate that a first layer “on” a second layer is directly on and in immediate contact with the second layer unless such is specifically stated; there may be a third layer or other structure between the first layer and the second layer on the first layer. The embodiments of a device or article described herein can be manufactured, used, or shipped in a number of positions and orientations. Persons skilled in the relevant art can appreciate that many modifications and variations are possible in light of the above teaching. Persons skilled in the art will recognize various equivalent combinations and substitutions for various components shown in the Figures. It is therefore intended that the scope of the invention be limited not by this detailed description, but rather by the claims appended hereto.

1. A surgical retractor system comprising:
a retractor body coupled to first, second, and third retractor arms;
a first retractor blade coupled to the first retractor arm, a second retractor blade coupled to the second retractor arm, and a third retractor blade coupled to the third retractor arm;
wherein the first retractor blade includes:
a blade body;
a first button;
a resilient member coupling the first button to the blade body;
a cam including a first cam plate including a first cam slot, a second cam plate including a second cam slot and a cam pin included within the first and second cam slots;
a rod coupling the first button to the cam;
wherein first blade is configured so: (a) depressing the first button drives the cam pin distally along a long axis of the first retractor blade, (b) in response to the cam pin being driven distally the cam pin travels simultaneously along the first and second cam slots; and (c) in response to the cam pin traveling simultaneously along the first and second cam slots, distal portions of the first and second cam plates move away from one another.

2. The surgical retractor system of claim 1, wherein the first blade is configured so: (a) releasing the first button drives the cam pin proximally along the long axis of the first retractor blade, (b) in response to the cam pin being driven proximally the cam pin travels simultaneously along the first and second cam slots; and (c) in response to the cam pin traveling simultaneously along the first and second cam slots, distal portions of the first and second cam plates move towards one another.

3. The surgical retractor system according to claim 2, wherein:
the first cam plate includes a first foot portion and the second cam plate includes a second foot portion;
the first foot portion includes a first void and the second foot portion includes a second void;
the first and second voids are configured to collectively surround a portion of a pedicle screw when the first button is released and the distal portions of the first and second cam plates move towards one another.

4. The surgical retractor system according to claim 3 comprising a pivot pin, wherein the first and second cam plates are pivotally coupled to one another via the pivot pin.

5. The surgical retractor system according to claim 4, wherein the first foot portion is included in a plane that is orthogonal to the long axis of the first retractor blade.

6. The surgical retractor system according to claim 2, wherein:
the first cam slot includes opposing ends coupled to one another by sidewalls that are primarily curved;
the second cam slot includes opposing ends coupled to one another by sidewalls that are primarily curved.

7. The surgical retractor system according to claim 2, wherein:
the first cam slot includes opposing ends coupled to one another by sidewalls that are primarily linear;
the second cam slot includes opposing ends coupled to one another by sidewalls that are primarily linear.

8. The surgical retractor system according to claim 2, wherein:
the first cam slot extends laterally as the first cam slot extends distally;
the second cam slot extends laterally as the second cam slot extends distally;
the first and second cam slots extend in opposing directions as the first and second cam slots extend distally and laterally.

9. The surgical retractor system according to claim 1, wherein:
the first retractor arm includes a lever and first and second portions;
the first and second portions are rotationally coupled to each other;
the first portion includes an arcuate row of first teeth and the second portion includes an arcuate row of second teeth keyed to securely couple with the first teeth;
in a first retractor arm orientation: (a) the lever does not directly contact the first portion, and (b) the first teeth do not directly contact the second teeth;
in a second retractor arm orientation: (a) the lever directly contacts the first portion, and (b) the first teeth directly contact the second teeth to prevent rotation of the first and second portions with regard to each other.

10. The surgical retractor system according to claim 1, wherein:
the first retractor arm includes first and second portions;
the first and second portions are rotationally coupled to each other;
the first portion includes an arcuate row of first teeth and the second portion includes an arcuate row of second teeth keyed to securely couple with the first teeth;
in a first retractor arm orientation the first teeth do not directly contact the second teeth;
in a second retractor arm orientation the first teeth directly contact the second teeth to prevent rotation of the first and second portions with regard to each other.

11. The surgical retractor system according to claim 1, wherein:
the first retractor arm includes a first actuator and first and second portions;
the first and second portions are rotationally coupled to each other;
the first portion includes an arcuate row of first teeth and the second portion includes an arcuate row of second teeth keyed to securely couple with the first teeth;
in a first retractor arm orientation: (a) the first actuator does not directly contact the first portion, and (b) the first teeth do not directly contact the second teeth;
in a second retractor arm orientation: (a) the first actuator directly contacts the first portion, and (b) the first teeth directly contact the second teeth to prevent rotation of the first and second portions with regard to each other.

12. The surgical retractor system according to claim 1, wherein:
the first retractor arm includes a first actuator and first and second portions;
the first and second portions are rotationally coupled to each other;
the first portion includes a first interface that is keyed to a second interface of the second portion;
in a first retractor arm orientation: (a) the first actuator does not directly contact the first portion, and (b) the first interface does not directly contact the second interface;
in a second retractor arm orientation: (a) the first actuator directly contacts the first portion, and (b) the first interface directly contacts the second interface to prevent rotation of the first and second portions with regard to each other.

13. The surgical retractor system according to claim 12, wherein the first portion is to rotate at least 90 degrees with respect to the second portion.

14. The surgical retractor system according to claim 12, wherein:
the second retractor arm includes an additional lever and additional first and second portions;
the additional first and second portions are rotationally coupled to each other;
the additional first portion includes an additional arcuate row of additional first teeth and the additional second portion includes an additional arcuate row of additional second teeth keyed to securely couple with the additional first teeth;
in an additional first retractor arm orientation: (a) the additional lever does not directly contact the additional first portion, and (b) the additional first teeth do not directly contact the additional second teeth;
in an additional second retractor arm orientation: (a) the additional lever directly contacts the additional first portion, and (b) the additional first teeth directly contact the additional second teeth to prevent rotation of the additional first and second portions with regard to each other.

15. The surgical retractor system according to claim 12, wherein
the first actuator includes a pivot point about which the first actuator pivots;
the first actuator has a perimeter, a first point on the perimeter is closer to the pivot point than a second point on the perimeter.

16. The surgical retractor system according to claim 1, wherein the second retractor arm is releasably coupled to the second retractor blade.

17. The surgical retractor system of claim 16 comprising a second button, wherein:
in a first blade orientation: (a) the second button is not depressed, and (b) the second retractor arm is locked to the second retractor blade;
in a second blade orientation the second retractor arm is not locked to the second retractor blade.

18. The surgical retractor system of claim 16 comprising a second actuator, wherein:
in a first blade orientation: (a) the second actuator is unactuated, and (b) the second retractor arm is locked to the second retractor blade;
in a second blade orientation the second retractor arm is not locked to the second retractor blade.

19. The surgical retractor system according to claim 17, wherein in the second blade orientation: (a) the second button is depressed, and (b) the second retractor arm is not locked to the second retractor blade.

20. The surgical retractor system according to claim 18, wherein in the second blade orientation: (a) the second actuator is actuated, and (b) the second retractor arm is not locked to the second retractor blade.

21. The surgical retractor system according to claim 19 wherein in the second blade orientation the second retractor arm is slidingly engaged with the second retractor blade.

22. The surgical retractor system according to claim 17 comprising a resilient member coupled to the second button, wherein the resilient member biases the second button into the first blade orientation.

23. The surgical retractor system of claim 16 comprising an additional second button, wherein:
in an additional first blade orientation: (a) the additional second button is not depressed, and (b) the first retractor arm is locked to the first retractor blade;
in an additional second blade orientation the first retractor arm is not locked to the first retractor blade.

24. The surgical retractor system according to claim 18 comprising:
a second actuator and a protuberance keyed to a void;
the second actuator includes one of the protuberance or the void and the second retractor blade includes another of the protuberance or the void;
in the first blade orientation the protuberance is located in the void.

25. The surgical retractor system according to claim 18 comprising:
a second actuator and a protuberance and a plurality of voids;
the second actuator includes one of the protuberance or the plurality of voids and the second retractor blade includes another of the protuberance or the plurality of voids;
in the first blade orientation the protuberance is located in one of the plurality of voids.

26. The surgical retractor system according to claim 18 comprising:
a second actuator and a plurality of protuberances and a void;
the second actuator includes one of the plurality of protuberances or the void and the second retractor blade includes another of the plurality of protuberances or the void;
in the first blade orientation one of the plurality of protuberances is located in the void.
